(19) United States
(12) Patent Application Publication
(10) Pub. No.: US 2025/0050119 A1
(43) Pub. Date: Feb. 13, 2025

(54) Garment Features for Therapy Electrode Pressure and/or Stabilization in a Wearable Medical Device

(71) Applicant: ZOLL Medical Corporation, Chelmsford, MA (US)

(72) Inventors: Sunaina Rustagi, Presto, PA (US); Christopher Lawrence Swenglish, Connellsville, PA (US); Milad Alizadeh-Meghrazi, Etobicoke (CA); Tony Chahine, Etobicoke (CA); Ladan Eskandarian, Etobicoke (CA)

(21) Appl. No.: 18/717,382

(22) PCT Filed: Dec. 5, 2022
(86) PCT No.: PCT/US2022/051875

§ 371 Date: Jun. 6, 2024

Related U.S. Application Data
(60) Provisional application No. 63/286,454, filed on Dec. 6, 2021.

(51) Int. Cl. A61N 1/39; A41D 1/00

(52) U.S. Cl. CPC A61N 1/3904; A41D 1/002; A61N 1/3906; A61N 1/3925

(57) ABSTRACT

A non-invasive wearable ambulatory cardiac defibrillator configured to improve therapy electrode contact with a patient's skin is provided. The defibrillator device includes a garment configured to be worn around the patient's torso, a sensing electrode attached to the garment and configured to sense electrical signal(s) at the surface of the patient's skin indicative of electrical activity of the patient's heart, and therapy electrodes attached to the garment and configured to deliver one or more defibrillation pulses to the patient. The therapy electrodes include at least two posterior therapy electrodes configured to be disposed on a posterior portion of the patient's body. The device further includes at least one strap attached to a back portion of the garment and exerting a normal force on the posterior therapy electrodes to exert a substantially uniform normal force over the surfaces of the posterior therapy electrodes and/or limit displacement of the posterior therapy electrodes.

### CROSS REFERENCE TO RELATED APPLICATION

This application claims priority to U.S. Provisional Patent Application No. 63/286,454, filed Dec. 6, 2021, the disclosure of which is hereby incorporated by reference in its entirety.

### FIELD OF THE DISCLOSURE

The present disclosure relates to wearable medical devices, and, more particularly, non-invasive wearable ambulatory cardiac defibrillators.

### BACKGROUND OF THE DISCLOSURE

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients who are at risk, have been hospitalized for, or otherwise are suffering from, adverse heart conditions can be prescribed a wearable cardiac monitoring and/or treatment device. As the wearable device is generally prescribed for continuous or near-continuous use (e.g., only to be removed when bathing), the patient wears the device during all daily activities such as walking, sitting, climbing stairs, resting or sleeping, and other similar daily activities. Maintaining continuous or near-continuous use of the device as prescribed can be important for monitoring patient progress as well as providing treatment to the patient if needed. Wearable defibrillator garments include one or more adjustment features intended for fitting the wearable defibrillator garment to the physiology of the patient to provide a comfortable fit. To ensure safe and reliable operation of the device while continuing to maintain a comfortable fit, it is desirable to ensure therapy electrode contact with the patient's skin.

### SUMMARY OF SOME OF THE EMBODIMENTS

Non-limiting examples of embodiments will now be described.

In an example, a non-invasive wearable ambulatory cardiac defibrillator is provided. The device comprises: a garment configured to be worn around a torso of a patient; at least one sensing electrode attached to the garment and configured to sense electrical signal(s) at the surface of the patient's skin indicative of electrical activity of the patient's heart; and two or more therapy electrodes attached to the garment and configured to deliver one or more defibrillation pulses to the patient. The two or more therapy electrodes comprise: an anterior therapy electrode configured to be disposed on an anterior portion of the patient's body; and at least two posterior therapy electrodes configured to be disposed on a posterior portion of the patient's body. The device comprises a controller in communication with the at least one sensing electrode and the therapy electrodes. The controller is configured to receive the electrical signal(s) from the at least one sensing electrodes and to cause delivery of the one or more defibrillation pulses from two or more therapy electrodes based on the controller detecting a cardiac arrhythmia in the received electrical signal(s). The device comprises at least one strap attached to a back portion of the garment. The at least one strap exerts a normal force on the at least two posterior therapy electrodes to exert a substantially uniform normal force over the surfaces of the at least two posterior therapy electrodes and/or limit displacement of the at least two posterior therapy electrodes.

The at least one strap can be attached to an external surface of the garment.

The at least one strap can comprise: a first strap extending from a right shoulder portion of the garment to a left waist portion of the garment; and a second strap extending from a left shoulder portion of the garment to a right waist portion of the garment such that the second strap crosses the first strap.

The at least one strap can be adjustable in length. Adjustment of the length of the at least one strap adjusts the normal force exerted on the at least two posterior therapy electrodes.

The at least one strap can comprise: a first strap extending from a right shoulder portion of the garment to a left shoulder portion of the garment; a second strap extending from the first strap to a right waist portion of the garment; and a third strap extending from the first strap to a left waist portion of the garment.

The first strap can comprise a curved portion extending over the at least one therapy electrode.

The at least one strap can comprise: a central strap extending at least partially over the at least one therapy electrode; a first strap extending from a right shoulder portion of the garment to the central strap; a second strap extending from a left shoulder portion of the garment to the central strap; a third strap extending from a right waist portion of the garment to the central strap; and a fourth strap extending from a left waist portion of the garment to the central strap.

The at least one strap can be disposed internally within the garment.

One of the garment and the at least one strap can comprise a plurality of loops. The other of the garment and the at least one strap can comprise a hook configured to engage any of the plurality of loops to adjust a tautness of the at least one strap.

The two or more therapy electrodes can be configured to deliver a biphasic shock to the patient.

The two or more therapy electrodes can be configured to deliver pacing pulses to the patient.

The controller can be configured to monitor for a ventricular fibrillation and/or a ventricular tachycardia event.

The controller can be configured to deliver a cardioversion shock to the patient via the two or more therapy electrodes.

The garment can be configured to be separable from the at least one sensing electrode and the two or more therapy electrodes.

The controller can be configured to generate ECG information from the electrical signal(s) received from the at least one sensing electrode and to cause delivery of the one or more therapeutic pulses from the at least one therapy electrode.

In an example, a non-invasive wearable ambulatory cardiac defibrillator is provided. The device comprises: a garment configured to be worn around a torso of a patient and defining an arm aperture for receiving the patient's arm; at least one sensing electrode attached to the garment and configured to sense electrical signal(s) at the surface of the patient's skin indicative of electrical activity of the patient's heart; and two or more therapy electrodes attached to the garment and configured to deliver one or more defibrillation pulses to the patient. The two or more therapy electrodes comprise: an anterior therapy electrode configured to be disposed on an anterior portion of the patient's body; and at least two posterior therapy electrodes configured to be disposed on a posterior portion of the patient's body. The device comprises a controller in communication with the at least one sensing electrode and the therapy electrodes. The controller is configured to receive the electrical signal(s) from the at least one sensing electrodes and to cause delivery of the one or more defibrillation pulses from the two or more therapy electrodes based on a cardiac arrhythmia being detected in the received electrical signal(s). The garment comprises an extension piece extending from an edge of the garment into the arm aperture. In some implementations, the extension piece is less elastic than a portion of the garment adjacent to the extension piece. In other implementations, the extension piece can be more elastic than a portion of the garment adjacent to the extension piece. Examples of relative elasticity of the extension piece when compared to the portion of the garment adjacent to the extension piece are described in further detail below.

The extension piece can extend upwardly along the edge of the garment to an upper end of the at least two posterior therapy electrodes.

The two or more therapy electrodes can be configured to deliver a biphasic shock to the patient.

The two or more therapy electrodes can be configured to deliver pacing pulses to the patient.

The controller can be configured to monitor for a ventricular fibrillation and/or a ventricular tachycardia event.

The controller can be configured to deliver a cardioversion shock to the patient via the two or more therapy electrodes.

The garment can be configured to be separable from the at least one sensing electrode and the two or more therapy electrodes.

The controller can be configured generate ECG information from the electrical signal(s) received from the at least one sensing electrode and to cause delivery of the one or more therapeutic pulses from the at least one therapy electrode.

In an example a non-invasive wearable ambulatory cardiac defibrillator is provided. The device comprises: a garment configured to be worn around a torso of a patient; at least one sensing electrode attached to the garment and configured to sense electrical signal(s) at the surface of the patient's skin indicative of electrical activity of the patient's heart; and two or more therapy electrodes attached to the garment and configured to deliver one or more defibrillation pulses to the patient. The two or more therapy electrodes comprise: an anterior therapy electrode configured to be disposed on an anterior portion of the patient's body; and at least two posterior therapy electrodes configured to be disposed on a posterior portion of the patient's body. The device comprises a controller in communication with the at least one sensing electrode and the therapy electrodes. The controller is configured to receive the electrical signal(s) from the at least one sensing electrodes and to cause delivery of the one or more defibrillation pulses from two or more therapy electrodes based on the controller detecting a cardiac arrhythmia in the received electrical signal(s). The device comprises a panel attached to a back portion of the garment at least partially over the at least two posterior therapy electrodes. The panel exerts a normal force on the at least two posterior therapy electrodes to exert a substantially uniform normal force over the surfaces of the at least two posterior therapy electrodes and/or limit displacement of the at least two posterior therapy electrodes. In some implementations, the panel can be made from a material that is less elastic (e.g., more stiff) than a material of the back portion of the garment. Examples of relative elasticity of the panel when compared to the portion of the garment adjacent to the panel are described in further detail below.

In some examples, the panel can be bonded to the back portion of the garment. In some examples, the panel can be attached to the back portion of the garment with an adhesive.

The panel can be attached to an external surface of the back portion of the garment.

The panel can be rectangular.

The panel can comprise a central section at least partially covering the at least one therapy electrode, a first strip extending from the central section to a right waist portion of the garment, and a second strip extending from the central section to a left waist portion of the garment.

The two or more therapy electrodes can be configured to deliver a biphasic shock to the patient.

The two or more therapy electrodes can be configured to deliver pacing pulses to the patient.

The controller can be configured to monitor for a ventricular fibrillation and/or a ventricular tachycardia event.

The controller can be configured to deliver a cardioversion shock to the patient via the two or more therapy electrodes.

The garment can be configured to be separable from the at least one sensing electrode and the two or more therapy electrodes.

The controller can be configured to generate ECG information from the electrical signal(s) received from the at least one sensing electrode and to cause delivery of the one or more therapeutic pulses from the at least one therapy electrode.

The first strip and the second strip can cover the at least one sensing electrode.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the disclosure.

Further features and other examples and advantages will become apparent from the following detailed description made with reference to the drawings.

FIG. 1 is a rear view of a non-invasive wearable ambulatory cardiac defibrillator according to an example of the present disclosure;

FIG. 2 is a rear view of a non-invasive wearable ambulatory cardiac defibrillator according to an example of the present disclosure;

FIG. 3 is a rear view of a non-invasive wearable ambulatory cardiac defibrillator according to an example of the present disclosure;

FIG. 4 is a side perspective view of a non-invasive wearable ambulatory cardiac defibrillator according to an example of the present disclosure;

FIG. 5 is a rear view of a non-invasive wearable ambulatory cardiac defibrillator according to an example of the present disclosure;

FIG. 6 is a rear view of a non-invasive wearable ambulatory cardiac defibrillator according to an example of the present disclosure;

FIG. 7 is a rear view of a non-invasive wearable ambulatory cardiac defibrillator according to an example of the present disclosure;

FIG. 8 is a force diagram of the non-invasive wearable ambulatory cardiac defibrillators of any of FIGS. 1-7, taken along section line X-X of FIG. 1;

FIG. 9 is a schematic view of a strap of the device of FIG. 4;

FIG. 10 is a schematic of an exemplary wearable cardiac monitoring and therapeutic medical device that can be used in connection with the present disclosure;

FIG. 11 is a front view of an exemplary support garment for the wearable cardiac monitoring and therapeutic medical device of FIGS. 1-10 as worn on a patient;

FIG. 12 is a rear view of the support garment of FIG. 11 as worn on a patient;

FIGS. 13A and 13B are a front view of an exemplary support garment and electrode assembly, respectively, for the wearable monitoring and therapeutic medical device that can be used in connection with the present disclosure;

FIG. 14 is a schematic of an exemplary wearable cardiac monitoring and therapeutic medical device that can be used in connection with the present disclosure;

FIG. 15A is a schematic drawing showing a front perspective view of an example monitor for the wearable medical device of FIG. 14;

FIG. 15B is a schematic drawing showing a rear perspective view of the example monitor of FIG. 15A;

FIG. 16 is a schematic diagram of functional components of the wearable medical device of FIG. 14;

FIG. 17A is a bottom view of a therapy electrode of the medical device of FIG. 1; and

FIG. 17B is a top view of the therapy electrode of FIG. 23A.

### DETAILED DESCRIPTION OF SOME OF THE EMBODIMENTS

As used herein, the singular forms of “a”, “an”, and “the” include plural referents unless the context clearly dictates otherwise.

As used herein, the terms “right”, “left”, “top”, and derivatives thereof shall relate to the disclosure as it is oriented in the drawing figures. However, it is to be understood that the disclosure can assume various alternative orientations and, accordingly, such terms are not to be considered as limiting. Also, it is to be understood that the disclosure can assume various alternative variations and stage sequences, except where expressly specified to the contrary. It is also to be understood that the specific devices and processes illustrated in the attached drawings and described in the following specification are examples. Hence, specific dimensions and other physical characteristics related to the embodiments disclosed herein are not to be considered as limiting.

For the purposes of this specification, unless otherwise indicated, all numbers expressing quantities of ingredients, reaction conditions, dimensions, physical characteristics, and so forth used in the specification and claims are to be understood as being modified in all instances by the term “about” or “approximately”. Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Also, it should be understood that any numerical range recited herein is intended to include all sub-ranges subsumed therein. For example, a range of “1 to 10” is intended to include any and all sub-ranges between and including the recited minimum value of 1 and the recited maximum value of 10; that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible.

Patients who are at risk, have been hospitalized for, or otherwise are suffering from, adverse heart conditions can be prescribed a wearable cardiac monitoring and/or treatment device. As the wearable device is generally prescribed for continuous or near-continuous use (e.g., only to be removed when bathing), the patient wears the device during all daily activities such as walking, sitting, climbing stairs, resting or sleeping, and other similar daily activities. Maintaining continuous or near-continuous use of the device as prescribed can be important for monitoring patient progress as well as providing treatment to the patient if needed.

In this disclosure, example features are described for adjusting the pressure and/or physically stabilizing one or more therapy electrodes on the patient's body.

In order to effectively and reliably deliver the treatment pulse, the therapy electrodes must be have reliable and continuous, contact with the patient's skin. In wearable defibrillator garments, inherent flexibility of the devices can result in the therapy electrodes partly or completely separating from skin contact. For example, therapy electrodes on the back (posterior) portion of wearable defibrillator garment are intended to contact the patient's back between and/or just below the shoulder blades. In patients with relatively prominent shoulder blades, the shoulder blades can tend to lift the wearable defibrillator garments away from the patient's upper back, potentially allowing separation of the therapy electrode from the patient's skin. Additionally, normal movements of the patient can relieve tension in the back portion of the wearable defibrillator garment, again potentially allowing separation of the therapy electrodes from the patient's skin. Wearable defibrillator garment features are described herein for adjusting the pressure and/or physically stabilizing one or more of the therapy electrodes on the patient's body, and for minimizing shifting and/or lifting of such therapy electrodes.

In examples described herein, relative fabric stiffness and/or elasticity properties can be measured in the following ways. In one example, stiffness (and/or fabric elasticity) can be measured, tested and/or recorded on a Universal Testing Machine or an INSTRON-4411 tensile test machine (CRE type) from INSTRON of Norwood, MA. In this example, the stiffness of two samples, one representing a first portion or component and the other representing a second portion or component can be characterized as two separate graphs of force (e.g., in pounds per inch) over distance. The slopes of the two graphs can be compared to determine relative stiffness of the two sample materials. In examples, testing of the samples can be performed in accordance with the procedures described in ASTM D 4964, Standard Test Method for Tension and Elongation of Elastic Fabrics (Constant-Rate-of-Extension Type Tensile Testing Machine). The fabric materials can be tested in accordance with other relevant testing standards, such as, ASTM D 5278, Standard Test Method for Elongation of Narrow Elastic Fabrics (Static-Load Testing), and ASTM D2731-21, Standard Test Method for Elastic Properties of Elastomeric Yarns (CRE Type Tensile Testing Machines). In another example, stretch and/or elasticity of a fabric can be measured in terms of a Poisson's Ratio, which is deformation of the specimen fabric material in directions perpendicular to the specific direction of loading. In this example, the ratio is characterized in terms of transverse strain over axial strain. In another example, stretch and/or elasticity of a fabric material can be measured in terms of “stretch and recovery,” where the specimen fabric material is stretched to a maximum limit without being deformed and such is measured in terms of a percentage per unit length. For example, such stretch can be along one axis or two orthogonal axes depending on the material and purpose of use of the material as described herein.

As summarized above, some examples disclosed herein are directed to a non-invasive wearable ambulatory cardiac defibrillator that improves therapy electrode contact with a patient's skin. These wearable medical devices are used in clinical or outpatient settings to monitor and/or record various electrocardiogram (ECG) and other physiological signals of a patient. Moreover, these wearable medical devices can analyze the ECG and other physiological signals to monitor for arrhythmias, and, in example devices described herein, provide treatment such as cardioverting, defibrillating, or pacing shocks/pulses via therapy electrodes in the event of life-threatening arrhythmias. Examples of cardiac monitoring and treatment devices that can implement the adjustable garment features and/or processes described herein includes wearable defibrillators, which are also called wearable cardioverter defibrillator (WCDs); and hospital wearable defibrillators (HWDs).

The therapy electrodes as described herein are configured to provide electrical treatment pulses/shocks. A controller in communication with the therapy electrodes can control the timing and electrical properties of the treatment shocks/pulses (e.g. cardioverting, defibrillating, or pacing shocks/pulses). Therapy electrodes can generally include a conductive bottom surface configured to establish an electrical interface with the patient's skin.

Additionally or alternatively, the therapy electrodes described herein can also be configured to dispense conductive gel onto the patient to improve the electrical interface between the conductive bottom surface and the skin. For example, each therapy electrode can include a plurality of openings on the conductive surface. On the side that is opposite to the side that interfaces with the patient's skin, the therapy electrode can include a plurality of gel reservoirs (e.g., in some implementations, about 2 to about 10 gel reservoirs, or 2 to 10 gel reservoirs, in some implementations, about 10 to about 20 gel reservoirs, or 10 to 20 gel reservoirs, or in some implementations, about 20 to about 100 gel reservoirs, or 20 to 100 gel reservoirs) disposed thereon. Each of the plurality of gel reservoirs includes a predetermined quantity of conductive gel, e.g., in some implementations, about 0.1 cubic-centimeter (cc) to about 2 cc, or 0.1 cc to 2 cc, in some implementations, about 2 cc to about 20 cc, or 2 cc to 20 cc, or in some implementations, about 20 cc to about 50 cc, or 20 cc to 50 cc. When the controller (as described in further detail below) determines that an electrical shock is warranted, the controller can cause the gel reservoirs to dispense the conductive gel in the interface between the conductive bottom surface and the patient's skin.

To effectively provide the treatment pulses described above, it is desirable that the therapy electrodes provided in the device appropriately contact the skin of the patient. For example, an acceptable contact pressure range at one or more electrode-to-skin interfaces can be selected based upon a predetermined minimum range of pressure that provides adequate contact between the electrodes and the patient's skin to facilitate essentially complete transmission of an electric shock/pulse from the electrodes to the patient. For example, an acceptable pressure range at one or more electrode-to-skin interfaces can include pressures, in some implementations, ranging from about 0.25 psi to about 0.62 psi, or from 0.25 psi to 0.62 psi, in some implementations ranging from about 0.4 psi to about 0.62 psi, or from 0.4 psi to 0.62 psi, or in some implementations ranging from about 0.5 psi to about 0.62 psi, or from 0.5 psi to 0.62 psi.

The devices described herein can include features to provide an acceptable range of pressure (e.g., ranging from about 0.25 psi to about 0.62 psi), and consequently a substantially uniform normal force acting substantially perpendicular to the patient's skin, to the therapy electrodes. This substantially uniform normal force can prevent displacement of the therapy electrodes and counteract separation of the electrode-to-skin interface so that the therapy electrodes can reliably deliver pulses/shocks to the patient. In particular, the devices described herein can include features that can improve electrode-to-skin contact, particularly with regard to posterior therapy electrodes configured to engage the patient's back. In some scenarios, e.g., certain patient body geometries, posterior therapy electrodes can be susceptible to separation from the patient's skin due to the physiology of the shoulder blades protruding relative to the portion of the back engaging the posterior therapy electrodes. Additionally, posterior therapy electrodes can be induced to separate from the patient's skin during routine articulation of the spine, neck, arms, and connected anatomy.

In examples, the devices described herein can include a wearable garment having at least one strap attached to a back portion of the garment. The at least one strap is configured to extend at least partially over the posterior therapy electrodes. In doing so, the at least one strap exerts a substantially uniform normal force over the externally facing surface(s) of the posterior therapy electrodes to improve contact between the posterior therapy electrodes and the patient's skin. The at least one strap thus counteracts any tendency of the posterior therapy electrodes to lift off or separate from the patient's skin, due to either a patient's physiological factors (e.g., prominent shoulder blades) or routine movement of the patient.

In examples, the devices described herein can include a garment having a panel attached to a back portion of the garment. The panel is configured to extend at least partially over the posterior therapy electrodes. In doing so, the panel exerts a substantially uniform normal force over the surfaces of the posterior therapy electrodes to improve contact between the posterior therapy electrodes and the patient's skin. The panel thus counteracts any tendency of the posterior therapy electrodes to lift off or separate from the patient's skin, due to either physiological factors (e.g. prominent shoulder blades) or routine movement of the patient. In examples, the panel can include additional sections or strips configured to extend over and apply pressure within an acceptable range (e.g., ranging from about 0.25 psi to about 0.62 psi, as described herein) to therapy electrodes of the device, thereby maintaining the electrode-to-skin interface of the therapy electrodes.

In examples, the devices described herein can include an extension piece extending into an arm aperture of the garment. The extension piece can be configured to impart a tension in the garment around the patient's torso, which consequently causes a substantially uniform normal force over the externally facing surface(s) of the posterior therapy electrodes to improve contact between the posterior therapy electrodes and the patient's skin. The extension piece thus counteracts any tendency of the posterior therapy electrodes to lift off or separate from the patient's skin, due to either physiological factors (e.g. prominent shoulder blades) or routine movement of the patient.

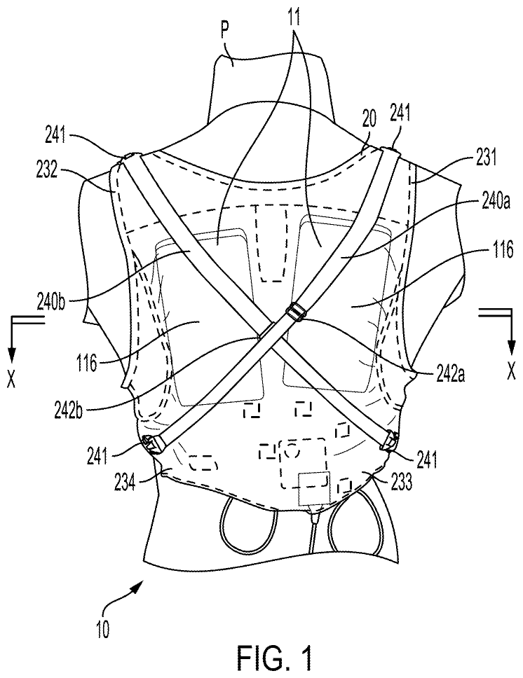

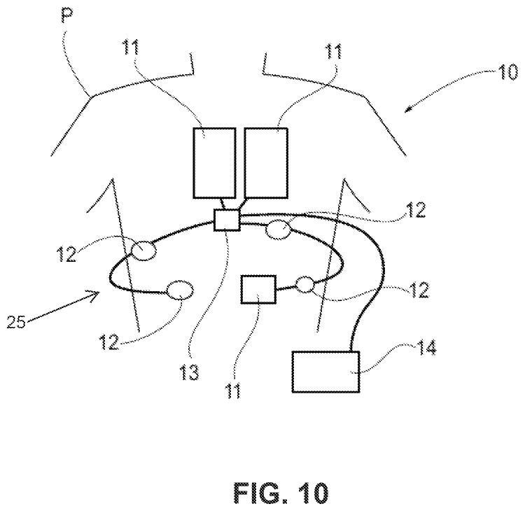

Referring now to the accompanying drawings, FIGS. 1-7 illustrate examples of non-invasive wearable ambulatory cardiac defibrillators 10 according to the present disclosure. In the various examples shown in FIGS. 1-7, components with like reference numerals can refer to like part from other examples. Each example of the device 10 can generally include at least two posterior therapy electrodes 11 configured to deliver one or more therapeutic pulses and/or shocks (e.g. a biphasic shock, pacing pulses, a cardioversion shock, etc.) to a patient P. The posterior therapy electrodes 11 are attached to a support garment 20 configured to be worn around the torso of the patient P. The support garment 20 holds the posterior therapy electrodes 11 at a clinically advantageous location with respect to the back of the patient P. The support garment 20 can include a right shoulder portion 231 configured to engage the right shoulder of the patient P, a left shoulder portion 232 configured to engage the left shoulder of the patient P, a right waist portion 233 configured to engage a right side of the waist of the patient P, and a left waist portion 234 configured to engage a left side of the waist of the patient P. Further details of the device 10, components thereof, and associated systems are shown and described herein with reference to FIGS. 10-17B.

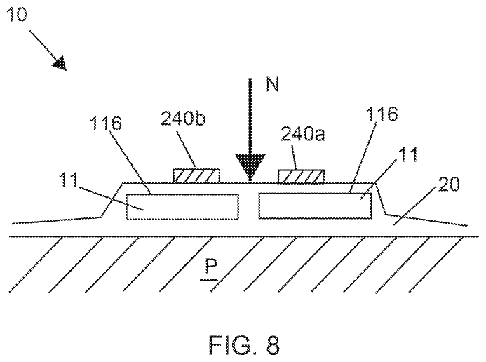

Referring now to the example shown in FIG. 1, the device 10 can include at least one strap, for example a first strap 240a and a second strap 240b, extending at least partially across the back of the garment 20. In one implementation, each of the straps 240a, 240b are mounted to an external surface of the garment 20 so as to be accessible to a user (e.g. the patient P or an assistant) when the garment 20 is being worn by the patient P. The straps 240a, 240b are configured to exert a normal force on the rear externally facing surface(s) 116 of the posterior therapy electrodes 11 to exert a substantially uniform normal force over the rear externally facing surface(s) 116 of the posterior therapy electrodes 11 and/or to limit displacement of the posterior therapy electrodes 11 relative to the back of the patient P. In particular, the straps are configured such that normal force(s) exerted by the straps 240a, 240b on the posterior therapy electrodes 11 act substantially perpendicular to a surface of the back of the patient P, thereby preventing the posterior therapy electrodes 11 from lifting away or otherwise separating from the patient P. A diagram of the normal force N is shown schematically in FIG. 8.

With continued reference to FIG. 1, the first strap 240a extends from the right shoulder portion 231 of the garment 20 to the left waist portion 234 of the garment 20. The second strap 240b extends from the left shoulder portion 232 of the garment 20 to the right waist portion 233 of the garment 20. In the example shown in FIG. 1, each of the shoulder portions 231, 232 and the waist portions 233, 234 of the garment 20 includes an anchor 241 for attachment to the straps 240a, 240b in the arrangement described. The anchors 241 include snaps, hooks, buttons, tabs and the like and can be configured for permanent or removable attachment to the straps 240a, 240b. In other examples, each end of the straps 240a, 240b can be permanently attached to the garment 20 with stitching, adhesive, and/or other permanent fastening fixtures. In examples, the second strap 240b can cross the first strap 240a, forming an “X” shape on the posterior of the garment 20.

With continued reference to FIG. 1, in some implementations, one or more of the straps 240a, 240b are adjustable in length. For example, the straps 240a, 240b can be configured such that adjustment of the length of the straps 240a, 240b alters the normal force N (shown in FIG. 8) exerted on the posterior therapy electrodes 11. For example, the straps 240a, 240b can be configured such that lengthening the straps 240a, 240b reduces the normal force N exerted on the posterior therapy electrodes 11, whereas shortening the straps 240a, 240b increases the normal force N exerted on the posterior therapy electrodes 11. The straps 240a, 240b can be configured such that, before or after the garment 20 is donned by the patient P, the straps 240a, 240b can be adjusted in length to achieve a desired level of normal force N exerted on the rear externally facing surface(s) 116 of the posterior therapy electrodes 11. In particular, the straps 240a, 240b can be configured so as to be adjustable in length to exert a substantially uniform normal force over the rear externally facing surface(s) 116 of the posterior therapy electrodes 11 and/or to limit displacement of the posterior therapy electrodes 11 relative to the back of the patient P. In some examples, either one or both of the straps 240a, 240b includes a respective slider 242a, 242b to allow for length adjustment.

In addition to adjusting the normal force N applied to the posterior therapy electrodes 11, the straps 240a, 240b can also be adjusted to realign or force the patient P into a physiologically improved posture.

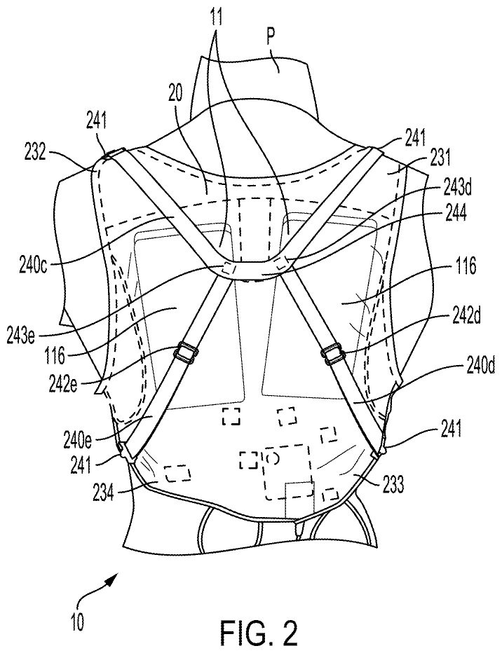

Referring now to the example shown in FIG. 2, in some implementations, the device 10 includes at least one strap (for example a first strap 240c, a second strap 240d, and a third strap 240e) extending at least partially across the back of the garment 20. The first strap 240c can extend from the right shoulder portion 231 of the garment 20 to the left shoulder portion 232 of the garment 20. The second strap 240d can extend from the first strap 240c to the right waist portion 233 of the garment 20. In particular, the second strap 240d can extend from a connection point 243d on the first strap 240c to the right waist portion 233 of the garment 20. The connection point 243d can be to the right of a centerline of the back of the patient P. Likewise, the third strap 240e can extend from the first strap 240c to the left waist portion 234 of the garment 20. In particular, the third strap 240e can extend from a connection point 243e on the first strap 240c to the left waist portion 234 of the garment 20. The connection point 243e can be to the left of a centerline of the back of the patient P. In some examples, the first strap 240c can include a curved portion 244 extending over the posterior therapy electrodes 11. The curved portion 244 can be induced as a result of the second strap 240d and the third strap 240e exerting tension on the first strap 240c. The tension exerted by the second strap 240d and the third strap 240e can also position the first strap 240c at a desired portion over the posterior therapy electrodes 11 in order to optimize the normal force applied to the rear externally facing surface(s) 116 of the posterior therapy electrodes 11. In some examples, the curved portion 244 can be stitched directly to the garment 20, such that tension in the second and third straps 240d, 240e is not required to maintain the shape of the curved portion 244.

The straps 240c, 240d, 240e are configured to exert a normal force N (shown in FIG. 8) on the rear externally facing surface(s) 116 of the posterior therapy electrodes 11 to exert a substantially uniform normal force over the rear externally facing surface(s) 116 of the posterior therapy electrodes 11 and/or to limit displacement of the posterior therapy electrodes 11 relative to the back of the patient P. In particular, the straps 240c, 240d, 240e can be configured such that the normal force exerted by the straps 240c, 240d, 240e on the rear externally facing surface(s) 116 of the posterior therapy electrodes 11 acts substantially perpendicular to a surface of the back of the patient P, thereby preventing the posterior therapy electrodes 11 from lifting away or otherwise separating from the patient P. In some examples, the second and third straps 240d, 240e include respective sliders 242d, 242e to adjust the length of the straps 240d, 240e, and thereby adjust the normal force N exerted on the posterior therapy electrodes 11. The second and third straps 240d, 240e can be configured such that adjustment of the second and third straps 240d, 240e is substantially the same as described herein in connection with the straps 240a, 240b of FIG. 1. While the first strap 240c is not adjustable in length in the example of FIG. 2, in other examples, the first strap 240c can include a slider similar to that of the second and third straps 240d, 240e to facilitate length adjustment of the first strap 240.

In addition to adjusting the normal force N applied to the posterior therapy electrodes 11, the straps 240c, 240d, 240f can also be adjusted to align or force the patient P into a physiologically improved posture.

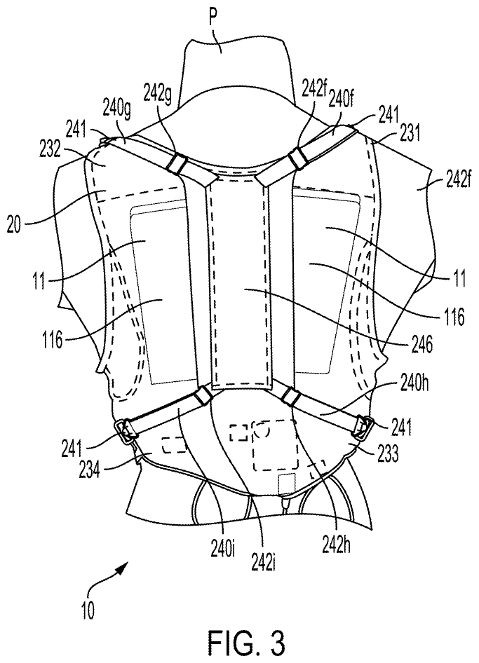

Referring now to the example shown in FIG. 3, in some implementations, the device 10 includes a central strap 246 extending at least partially over the posterior therapy electrodes 11. The central strap 246 can be rectangular in shape as shown in FIG. 3, although other shapes are also within the scope of this disclosure. The central strap 246 can be attached to the garment 20 by one or more supporting straps (for example a first strap 240f, a second strap 240g, a third strap 240h, and a fourth strap 240i), which can be adjustable as described herein to allow for positioning of the central strap 246. The first strap 240f can extend from the central strap 246 to the right shoulder portion 231 of the garment 20. The second strap 240g can extend from the central strap 246 to the left shoulder portion 232 of the garment 20. The third strap 240h can extend from the central strap 246 to the right waist portion 233 of the garment 20. The fourth strap 240i can extend from the central strap 246 to the left waist portion 234 of the garment 20. The central strap 246 can be substantially wider than the first strap 240f, the second strap 240g, the third strap 240h, and the fourth strap 240i. For example, the central strap 246 can be at least twice as wide as the first strap 240f, the second strap 240g, the third strap 240h, and the fourth strap 240i. The central strap 246 can be an elastic material in some examples, or a substantially inelastic material in other examples.

The straps 246, 240f, 240g, 240h, 240i are configured to exert a normal force N (shown in FIG. 8) on the rear externally facing surface(s) 116 of the posterior therapy electrodes 11 to exert a substantially uniform normal force over the rear externally facing surface(s) 116 of the posterior therapy electrodes 11 and/or to limit displacement of the posterior therapy electrodes 11 relative to the back of the patient P. In particular, the straps 246, 240f, 240g, 240h, 240i can be configured such that the normal force exerted by the straps 246, 240f, 240g, 240h, 240i on the posterior therapy electrodes 11 acts substantially perpendicular to a surface of the back of the patient P, thereby preventing the posterior therapy electrodes 11 from lifting away or otherwise separating from the patient P. In some examples, the first, second, third, and fourth straps 240f, 240g, 240h, 240i can include respective sliders 242f, 242g, 242h, 242i to adjust the length of the straps 240f, 240g, 240h, 240i, and thereby adjust the position of the central strap and the normal force N exerted on the posterior therapy electrodes 11. The first, second, third, and fourth straps 240f, 240g, 240h, 240i can be configured such that adjustment of the first, second, third, and fourth straps 240f, 240g, 240h, 240i is substantially the same as described herein in connection with the straps 240a, 240b of FIG. 1.

In addition to adjusting the normal force N applied to the posterior therapy electrodes 11, the straps 240f, 240g, 240h, 240i can also be adjusted to adjust or force the patient P into a physiologically improved posture.

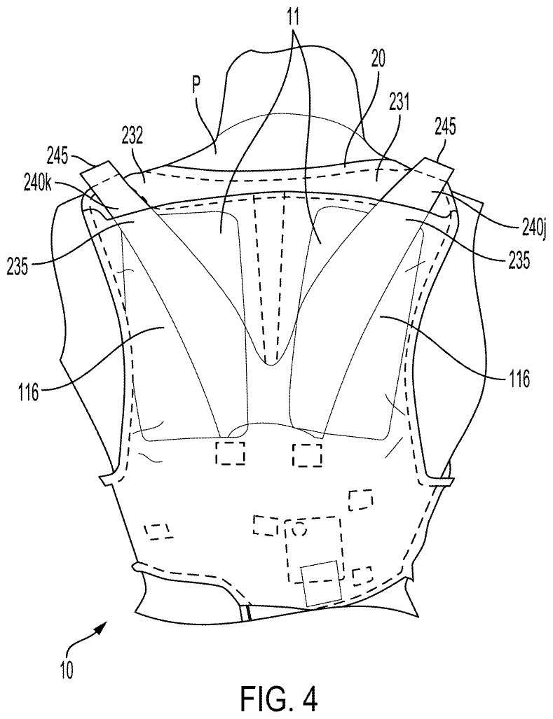

Referring now to the example shown in FIG. 4, in some implementations, the device 10 includes at least one strap (for example a first strap 240j and a second strap 240k) attached internally within the garment 20. The first and second straps 240j, 240k can be stitched internally to the garment 20 and extend at least partially over the posterior therapy electrodes 11. The first strap 240j can extend to the right shoulder portion 231 of the garment 20, and the second strap 240k can extend to the left shoulder portion 232 of the garment 20. The first strap 240j can extend to an external surface of the garment 20 through an aperture 235 at or near the right shoulder portion 231 so that the first strap 240j can be accessed for adjustment. Similarly, the second strap 240k can extend to an external surface of the garment 20 through an aperture 235 at or near the left shoulder portion 232 so that the second strap 240k can be accessed for adjustment. The straps 240j, 240k are configured to exert a normal force N (shown in FIG. 8) on the posterior therapy electrodes 11 to exert a substantially uniform normal force N over the rear externally facing surface(s) 116 of the posterior therapy electrodes 11 and/or to limit displacement of the posterior therapy electrodes 11 relative to the back of the patient P. In particular, the straps 240j, 240k can be configured such that the normal force N exerted by the straps 240j, 240k on the rear externally facing surface(s) 116 of the posterior therapy electrodes 11 acts substantially perpendicular to a surface of the back of the patient P, thereby preventing the posterior therapy electrodes 11 from lifting away or otherwise separating from the patient P.

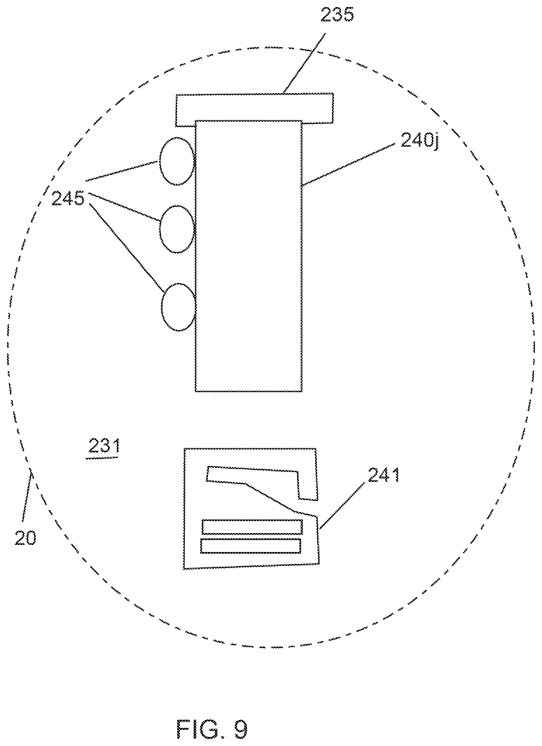

Additionally, the first and second straps 240j, 240k can be adjustable in length to allow for adjustability of the normal force exerted on the posterior therapy electrodes 11. As shown in FIG. 9, in some examples, the first strap 240j can include a plurality of loops 245 configured for connection to the anchor 241 on the right shoulder portion 231 of the garment 20. In the illustrated example, the anchor 241 is shown as a G-hook, though other connection devices are within the scope of the present disclosure. The anchor 241 can be attached to any of the plurality of the loops 245 to adjust the length of the first strap 240j, thereby adjusting the normal force N (shown in FIG. 8) exerted on the posterior therapy electrodes 11. In a similar manner, the second strap 240k can include a plurality of loops 245 for adjustable attachment to the anchor 241 on the left shoulder portion 232 of the garment. In other examples not shown, the plurality of the loops 245 are provided on the garment 20 and the corresponding anchors 241 are provided on the straps 240j, 240k as an alternative arrangement for achieving adjustability of the straps 240j, 240k.

In addition to adjusting the normal force N applied to the rear externally facing surface(s) 116 of the posterior therapy electrodes 11, the straps 240j, 240k can also be adjusted to force the patient P into a physiologically improved posture.

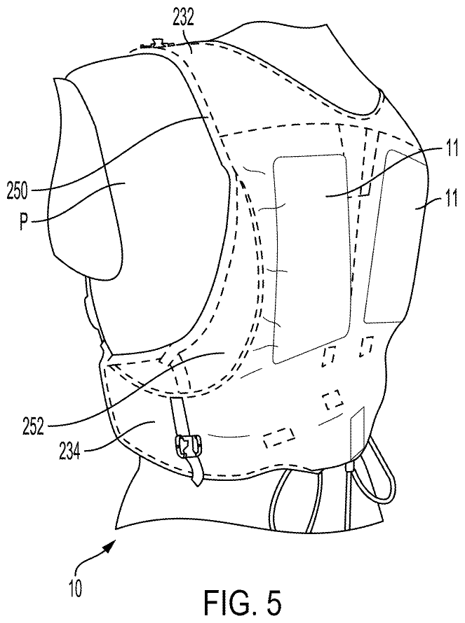

Referring now to the example shown in FIG. 5, in some implementations, the garment 20 includes an arm aperture 250 through which the arm of the patient extends. An arm aperture 250 on the left side of the garment 20 (as illustrated in FIG. 5) can be defined generally between the left shoulder portion 232 and the left waist portion 234. The garment 20 in the examples of FIG. 5 includes an extension piece 252 extending into the arm aperture 250. In some examples, the extension piece 252 can be generally arcuate in shape. The extension piece 252 can extend upwardly along the edge of the arm aperture 250 to an upper end of the posterior therapy electrodes 11.

The extension piece 252 can be made from a material, such as a fabric material, that is less elastic than the portion of the garment 20 (e.g. the left waist portion 234) adjacent the extension piece 252. The lesser elasticity (i.e. greater stiffness) of the extension piece 252 relative to adjacent portions of the garment 20 creates tension that increases the normal force N (shown in FIG. 8) exerted on the posterior therapeutic electrodes 11. In some examples, the extension piece 252 has a stiffness, as determined under any of the applicable ASTM standards described herein, of 25%-150% the stiffness of the adjacent portion of the garment 20, or of 150%-200% the stiffness of the adjacent portion of the garment 20, or of 200%-300% the stiffness of the adjacent portion of the garment 20. In some examples, one or more sections of stitching and/or fastening material that bonds the extension piece 252 to the adjacent portion of the garment 20 also affects the relative stiffness and/or elasticity behavior(s) of the extension piece 252 when compared to the adjacent portion of the garment 20. In this regard, the overall effect of the techniques and systems described herein is based on the combined garment 20 achieving the objectives of the present disclosure. For example, such overall effect can be based on the garment applying an acceptable pressure range at one or more therapy electrode-to-skin interfaces to include pressures ranging from about 0.25 psi to about 0.62 psi, or 0.25 psi to 0.62 psi, as described above. In some examples, the extension piece 252 can be less elastic than the adjacent portion of the garment 20 because the extension piece 252 is stitched to the garment 20 in a partially pre-stretched state, such that the a greater force is required to further stretch the extension piece 252 than to stretch the adjacent portion of the garment 20. The tension induced by the extension piece 252 causes the garment 20 to exert a substantially uniform normal force N over the rear externally facing surface(s) 116 of the posterior therapy electrodes 11 and/or to limit displacement of the posterior therapy electrodes 11 relative to the back of the patient P.

The extension piece 252 can be used alone as shown in FIG. 5 or can be used in conjunction with any of the examples of FIG. 1-4, 6, or 7. In addition to stabilizing the posterior therapy electrodes 11, the extension piece 252 can also improve the fit of the garment 20 against the patient's skin, and can make the garment 20 feel more secure.

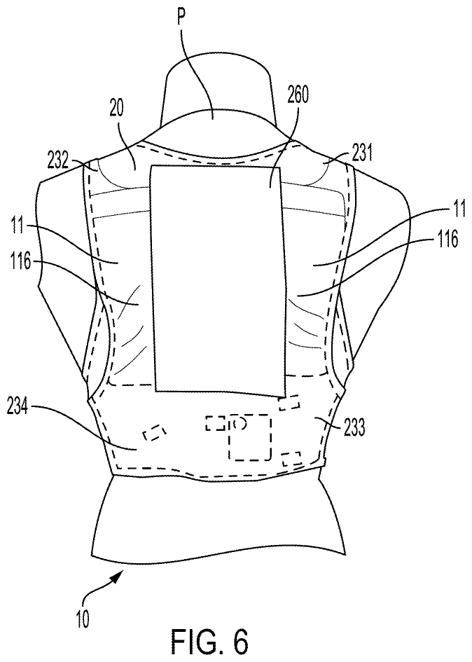

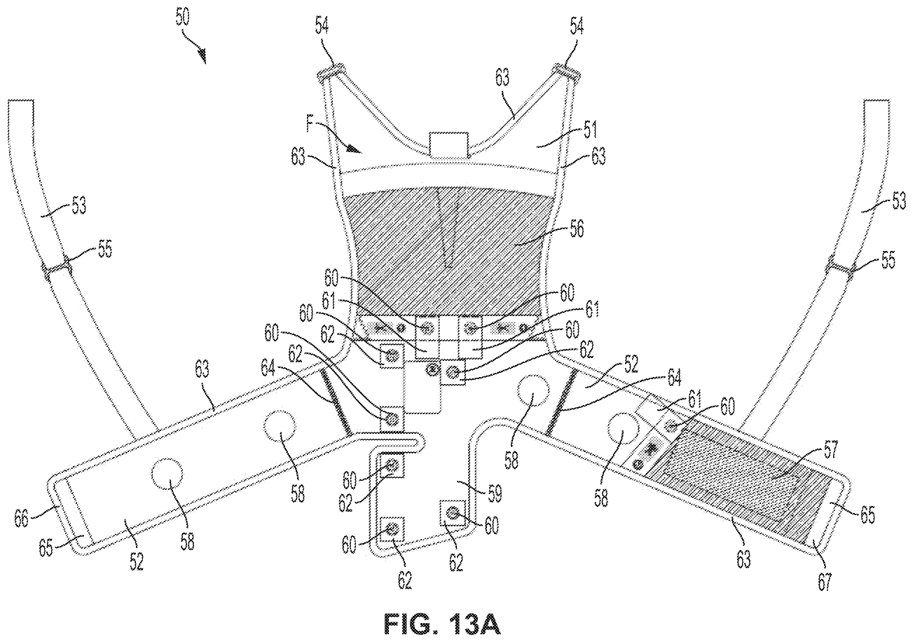

Referring now to the example shown in FIG. 6, in some implementations, the device 10 includes a panel 260 attached to a back portion of the garment 20 extending at least partially over the posterior therapy electrodes 11. The panel 260 can be attached to an external surface of the garment 20 as shown in FIG. 12, although in other examples the panel 260 can be internally disposed in the garment 20. The panel 260 can be rectangular, though other shapes are also within the scope of this disclosure. The panel 260 can be attached to the garment 20 by an adhesive, thermal bonding, or other compatible joining process. In some example implementations, the panel 260 incorporates materials such as HIGH RECOVERY, 3412, TRUFIT, OT100, OT523, NYL100, EX03900, EX00523, SLD3900, and FLOWFLEX3900 from Bemis Associates Inc., of Shirley, MA. The panel 260 can be made from a material that is less elastic (i.e. more stiff) than the portion of the garment 20 to which the panel 260 is attached. In some examples, the panel 260 has a stiffness, as determined under any of the applicable ASTM standards described herein, of 150%-200% the stiffness of the adjacent portion of the garment 20, or of 200%-300% the stiffness of the adjacent portion of the garment 20. In some examples, the panel 260 can be substantially non-stretchable under the forces to which it is subjected during normal use by the patient P. The panel 260 thus provides a reinforcing structure that limits or prevents stretching and movement of the garment 20 in the region of the posterior therapy electrodes 11. The panel 260 thus causes the garment 20 to exert a substantially uniform normal force N over the rear externally facing surface(s) 116 surfaces of the posterior therapy electrodes 11 and/or to limit displacement of the posterior therapy electrodes 11 relative to the back of the patient P. The panel 260 can particularly prevent the upper portion of the posterior therapy electrodes 11 for pulling away from the patient's shoulder blades.

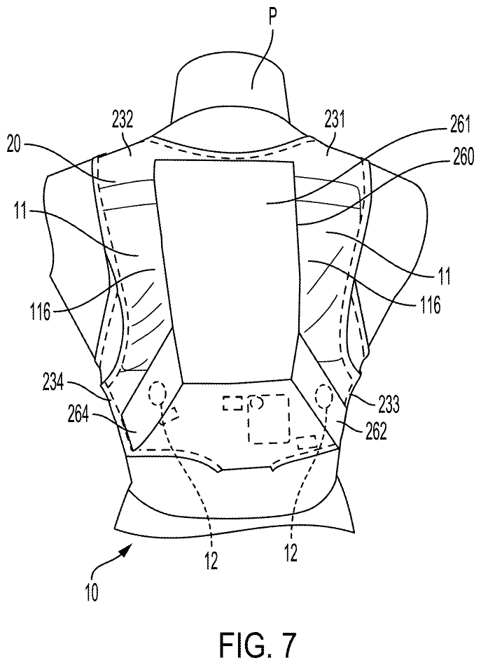

Referring now to the example shown in FIG. 7, in some implementations, the panel 260 includes a central section 261 at least partially covering the posterior therapy electrodes 11. The central section 261 provides a reinforcing structure that limits or prevents stretching and movement of the garment 20 in the region of the posterior therapy electrodes 11, as described in connection with FIG. 6. The panel 260 can further include a first strip 262 and a second strip 264 extending from the central section 261 towards a waist portion of the garment 20. In particular, the first strip 262 can extend from a bottom right corner of the central section 261 to the right waist portion 233 of the garment 20. The second strip 264 can extend from a bottom left corner of the central section 261 to the left waist portion 234 of the garment 20. The first and second strips 262, 264 can be formed integrally with the central section 261 or as separate pieces. The first and second strips 262, 264 can be made of the same material as the central section 261, or the first and second strips 262, 264 can be formed of a different material than the central section 261. The first and second strips 262, 264 can be particularly configured to cover, stabilize and prevent flipping of the sensing electrodes 12 positioned between the first and second strip 262, 264 and the patient's skin during patient movement.

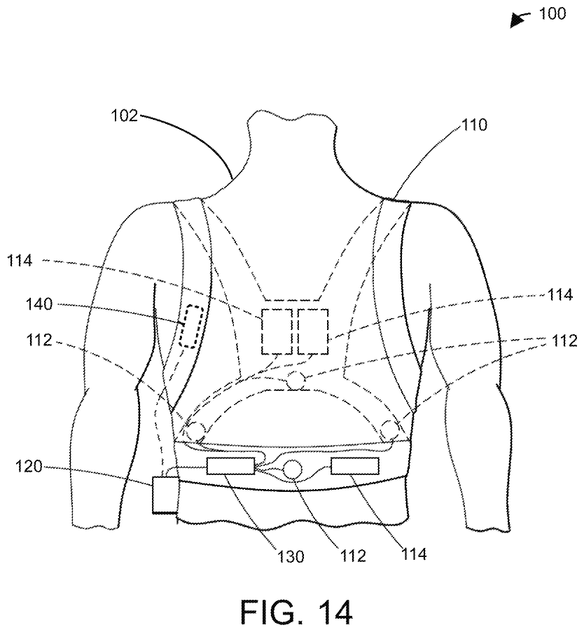

FIG. 10 illustrates an exemplary wearable medical device 10, such as a wearable defibrillator, that is external, non-invasible, ambulatory, and wearable by a patient P and is configured to implement one or more configurations described herein. For example, the wearable medical device 10 can correspond to and/or include features of the examples shown in FIGS. 1-7. The wearable medical device 10 can be an external or non-invasive medical device, e.g., the device 10 configured to be located substantially external to the patient P. The wearable defibrillator 10 can be worn or carried by an ambulatory patient P. According to one example of the present disclosure, the wearable defibrillator 10 is used as an ambulatory cardiac monitoring and treatment device within a monitoring and treatment system according to the present disclosure. FIGS. 14-16, discussed in detail below, illustrate in further detail an exemplary wearable medical device 100 in accordance with the present disclosure.

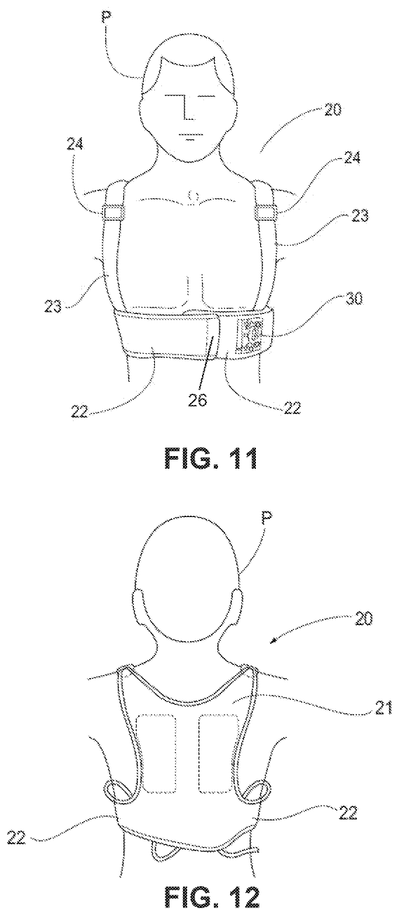

In accordance with one or more examples, a support garment 20 incorporating the features described herein is provided to keep the electrodes 11 and sensing electrodes 12 in place against the patient's body while remaining comfortable during wear. FIGS. 11 and 12 illustrate such a support garment 20 in accordance with an example of the present disclosure.

In order to obtain a reliable ECG signal so that the monitor can function effectively and reliably, the sensing electrodes 12 must be in the proper position and in good contact with the patient's skin. The electrodes 12 need to remain in a substantially fixed position and not move excessively or lift off the skin's surface. If there is excessive movement or lifting, the ECG signal will be adversely affected with noise and can cause problems with the arrhythmia detection and in the ECG analysis and monitoring system. Similarly, in order to effectively deliver the defibrillating energy, the therapy electrodes 11 are configured to remain in position and in contact with the patient's skin.

In accordance with one or more examples, the support garment 20 as described in this disclosure can provide comfort and functionality under circumstances of human body dynamics, such as bending, twisting, rotation of the upper thorax, semi-reclining, and lying down. These are also positions that a patient can assume if he/she were to become unconscious due to an arrhythmic episode. The design of the garment 20 is generally such that it minimizes bulk, weight, and undesired concentrations of force or pressure while providing the necessary radial forces upon the treatment and sensing electrodes 11, 12 to ensure device functionality. A wearable defibrillator monitor 14 can be disposed in a support holster operatively connected to or separate from the support garment 20. The support holster can be incorporated in a band or belt worn about the patient's waist or thigh.

As shown in FIGS. 11 and 12, in some implementations, the support garment 20 as described in this disclosure is provided in the form of a vest or harness having a back portion 21 and sides extending around the front of the patient P to form a belt 22. The ends of the belt 22 are connected at the front of the patient P by a closure 26, which can comprise one or more clasps. Multiple corresponding closures can be provided along the length of the belt 22 to allow for adjustment in the size of the secured belt 22 in order to provide a more customized fit to the patient P. The support garment 20 can further include two straps 23 connecting the back portion 21 to the belt 22 at the front of the patient P. The straps 23 have an adjustable size to provide a more customized fit to the patient P. The straps 23 can be provided with sliders 24 to allow for the size adjustment of the straps 23. The straps 23 can also be selectively attached to the belt 22 at the front of the patient P. The support garment 20 can be comprised of an elastic, low spring rate material that stretches appropriately to keep the electrodes 11, 12 in place against the patient's skin while the patient P moves and is lightweight and breathable. For example, the support garment 20 can have elastic, low spring rate material composition based on a fiber content of about 20% elastic fiber, about 32% polyester fiber, and up to about 48% or more of nylon or other fiber.

In accordance with one or more examples, the support garment 20 as described in this disclosure is formed from an elastic, low spring rate material and constructed using tolerances that are considerably closer than those customarily used in garments. The materials for construction are chosen for functionality, comfort, and biocompatibility. The materials can be configured to wick perspiration from the skin. The support garment 20 can be formed from one or more blends of nylon, polyester, and spandex fabric material. Different portions or components of the support garment 20 can be formed from different material blends depending on the desired flexibility and stretchability of the support garment 20 and/or its specific portions or components. For instance, the belt 22 of the support garment 20 can be formed to be more stretchable than the back portion 21. According to one example, the support garment 20 as described in this disclosure is formed from a blend of nylon and spandex materials, such as a blend of about 77% nylon and about 23% spandex. According to another example, the support garment 20 as described in this disclosure is formed from a blend of nylon, polyester, and spandex materials, such as about 40% nylon, about 32% polyester, and about 14% spandex. According to another example, the support garment 20 as described in this disclosure is formed from a blend of polyester and spandex materials, such as about 86% polyester and about 14% spandex or about 80% polyester and about 20% spandex. For example, the nylon and spandex material is configured to be aesthetically appealing, and comfortable, e.g., when in contact with the patient's skin. Stitching within the support garment 20 can be made with industrial stitching thread. According to one example, the stitching within the support garment 20 is formed from a cotton-wrapped polyester core thread.

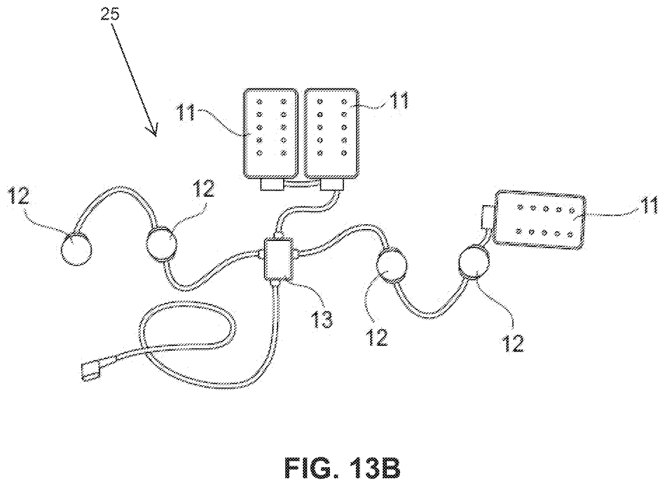

FIGS. 13A and 13B illustrate an exemplary support garment 50 according to the present disclosure. The support garment 50 incorporates additional improvements for enhancing the patient's experience in wearing the support garment for an extended period of time. The support garment examples provided herein promote comfort, aesthetic appearance, and ease of use or application for older patients, or patients with physical infirmities and/or who are physically challenged, including patients with rheumatic conditions, patients with arthritis, and/or patients with autoimmune or inflammatory diseases that affect joints, tendons, ligaments, bones, and muscles of the arm and hand. Patients afflicted with such conditions can properly and/or correctly don the garments described herein. Features of the support garments can also help minimize the time needed by patients to assemble, don or remove the support garment. Further, patients benefit from such features, which can facilitate longer wear times, better patient compliance, and improve the reliability of the detected physiological signals and treatment of the patient. These features promote ease of use, comfort and an aesthetic appearance for such patient populations. For example, the garments described herein generally follow design principles as noted below (e.g., similar to those prescribed in the Arthritis Foundation Guidelines).
Removing, donning, and assembling the garment and associated components do not require fine motor control or simultaneous actions,
Replacing electrodes and other components is possible for patients with limited reach and strength,
The garment and/or components include surface and/or textural aspects that makes the garment and/or components easy to grip and control.
The garment and/or components include features designed to minimize simultaneous actions such as depressing and pulling,
The garment and/or components include features to provide positive feedback (for example, “snap”, “click”, among others).

These features can encourage patients to wear the support garment and associated medical device for longer and/or continuous periods of time with minimal interruptions in the periods of wear. For example, by minimizing interruptions in periods of wear and/or promoting longer wear durations, patients and caregivers can be assured that the device is providing desirable information about as well as protection from adverse cardiac events such as ventricular tachycardia and/or ventricular fibrillation, among others. Moreover, when the patient's wear time and/or compliance is improved, the device can collect information on arrhythmias that are not immediately life-threatening, but can be useful to monitor for the patient's cardiac health. Such arrhythmic conditions can include onset and/or offset of bradycardia, tachycardia, atrial fibrillation, pauses, ectopic beats bigeminy, trigeminy events among others. For instance, episodes of bradycardia, tachycardia, or atrial fibrillation can last several minutes and/or hours. The support garments herein provide features that encourage patients to keep the device on for longer and/or uninterrupted periods of time, thereby increasing the quality of data collected about such arrhythmias. Additionally, features as described herein promote better patient compliance resulting in lower false positives and noise in the physiological signals collected from ECG electrodes and other sensors disposed within the support garment. For example, when patients wear the device for longer and/or uninterrupted periods of time, the device tracks cardiac events and distinguishes such events from noise over time.

The improvements incorporated in the support garment 50 can provide comfort and wearability to the patient by utilizing softer materials for at least some of the components of the support garment and by utilizing materials and construction features that are less likely to dig into and/or rub on the patient's skin in a painful or irritating manner.

In accordance with one or more examples, the support garment 50 is provided to keep the electrodes 11, 12 of an electrode assembly 25 associated with a wearable cardiac therapeutic device in place against the patient's body while remaining comfortable to wear. In particular, the electrode assembly 25 can include a plurality of ECG sensing electrodes 12 configured to sense ECG signals regarding a cardiac function of the patient and a plurality of therapy electrodes 11 configured to deliver transcutaneous defibrillation shocks or transcutaneous pacing pulses to the patient's heart. Examples of the wearable cardiac therapeutic devices in which the support garment 50 can be utilized include the wearable medical device 14 described above with reference to FIG. 10 and the wearable medical device 100 described in detail below with reference to FIGS. 14-16.

As shown in FIGS. 13A and 13B, in some examples, the support garment 50 is provided in the form of a vest or harness having a back portion 51 and sides extending around the front of the patient to form a belt 52. The ends 66, 67 of the belt 52 are connected at the front of the patient by a closure mechanism 65. The support garment 50 can further include straps as discussed in detail herein connecting the back portion 51 to the belt 52 at the front of the patient. The straps 53 have an adjustable size to provide a more customized fit to the patient. The straps 53 can also be selectively attached to the belt 22 at the front of the patient. The support garment 50 can be comprised of an elastic, low spring rate fabric material F that stretches appropriately to keep the electrodes 11, 12 in place against the patient's skin and is lightweight and breathable. The component materials of the fabric material F can be chosen for functionality, comfort, and biocompatibility. The component materials can be configured to wick perspiration from the skin. For example, the fabric material F can comprise a tricot fabric, the tricot fabric comprising nylon and spandex materials. The tricot fabric can comprise about 65% to about 90% nylon material, more particularly about 70% to about 85% nylon material, more particularly about 77% nylon material. It is to be appreciated that the fabric material F chosen for the support garment 50 can be comprised of any suitable materials or combinations of materials.

The support garment 50 as described in this disclosure can be configured for one-sided assembly of the electrode assembly 25 onto the support garment 50 such that the support garment 50 does not need to be flipped or turned over in order to properly position the therapy electrodes 11 and the sensing electrodes 12 on the support garment 50. The inside surface of the back portion 51 of the support garment 50 includes pocket(s) 56 for receiving one or two therapy electrodes 11 to hold the electrode(s) 11 in position against the patient's back. The pocket 56 is made from a non-elastic, conductive mesh fabric designed to isolate the metallic therapy electrode(s) 11 from the skin of the patient while allowing a conductive gel that can be automatically extruded from the electrode(s) 11 to easily pass through. The forces applied to the electrode(s) 11 by the fabric, in addition to the use of the conductive gel, can help ensure that proper contact and electrical conductivity with the patient's body are maintained, even during body motions. The fabric material of the pocket(s) 56 also maintains electrical contact between the electrode(s) 11 through the mesh material before the conductive gel is dispensed, which allows for monitoring of the therapy electrode(s) 11 to ensure that the electrode(s) 11 are positioned against the skin such that a warning can be provided by the wearable defibrillator 14 if the therapy electrode(s) 11 is not properly positioned. Another pocket 57 made from the same non-elastic, conductive mesh fabric is included on an inside surface of the belt 52 for receiving a therapy electrode 11 and holding the electrode 11 in position against the patient's left side. According to one example, the pockets 56, 57 are formed from an electrically conductive knit material. The material of the pockets 56, 57 can have a metal coating, such as a silver coating, applied thereto to provide electrical conductivity. The pockets 56, 57 can be closed by any suitable closure device 60, such as a hook and look fastener.

The back portion 51 and the belt 52 of the support garment 50 can further incorporate attachment points 58 for supporting the sensing electrodes 12 in positions against the patient's skin in spaced locations around the circumference of the patient's chest. The attachment points 58 can include hook-and-loop fasteners for attaching electrodes 12 having a corresponding fastener disposed thereon to the inside surface of the belt 52. The support garment 50 can further be provided with a flap 59 extending from the back portion 51. The flap 59 and the back portion 51 include a closure device 60 such as a hook and loop fastener for connecting the flap 59 to the inside surface of the back portion 51 in order to define a pouch or pocket for holding a distribution box 13 of the electrode assembly 25. The outer surface of the belt 52 can incorporate a schematic 30 (shown in FIG. 2) imprinted on the fabric for assisting the patient or medical professional in assembling the electrode assembly 25 onto the support garment 50.

Further discussion of the additional improvements incorporated into the support garment 50 for enhancing the patient's experience in wearing the support garment 50 for an extended period of time according to one or more examples of the present disclosure is provided below with reference to FIGS. 1-7 and 13A-13B.

With reference to FIGS. 13A and 13B, according to an example of the present disclosure, the support garment 50 can be incorporated into a wearable cardiac therapeutic device with improved fasteners for fastening and supporting electrodes on the support garment 50.

The device includes a plurality of ECG sensing electrodes 12 configured to sense ECG signals regarding a cardiac function of the patient and the support garment 50 configured to support and hold the plurality of ECG sensing electrodes 12 against the patient's body. The device includes a plurality of therapy electrodes 11 configured to deliver transcutaneous defibrillation shocks, transcutaneous cardioversion shocks, and/or transcutaneous pacing pulses to the patient's heart. The support garment 50 can be configured to support and hold the plurality of therapy electrodes 11 against the patient's body in accordance with implementations described herein. The support garment 50 includes a plurality of fasteners/attachment points 58 on an inside surface thereof for fastening and supporting the plurality of ECG sensing electrodes 12 on the support garment 50.

Each of the plurality of fasteners/attachment points 58 can include a hook and loop fastener patch affixed to a predetermined location on the inside surface of the support garment 50. Each of the plurality of ECG sensing electrodes 12 includes a corresponding hook and loop fastener patch configured to connect to a respective hook and loop fastener patch on the support garment 50.

The hook and loop fastener patches are configured to facilitate alignment and assembly of the respective ECG sensing electrodes 12 on the support garment 50 and to provide for fastening and support for the respective ECG sensing electrodes 12 on the support garment independent of the rotational orientation of the respective ECG sensing electrodes 12. This provides for easier assembly of the ECG sensing electrodes 12 on the support garment 50 and less error with respect to the assembly of the ECG sensing electrodes 12 on the support garment 50 resulting from misalignment of on the ECG sensing electrodes 12 with the hook and loop fastener patch of the fasteners/attachment points 58 on the support garment 50.

According to an example, each of the hook and loop fastener patches has a length of about 0.5″ to about 3.0″ to about and a width of about 0.5″ to about 3.0″. According to another example, each of the circular hook-and-loop fastener patches has a length and width of about 1.25″, respectively. It is to be appreciated that the hook and loop fastener patches can be of any suitable size.

According to an example, the hook and loop fastener patch can comprise a nylon, polyester, or polypropylene material. It is to be appreciated that the hook and loop fastener patch can comprise any suitable materials.

According to an example, the hook and loop fastener patch are permanently affixed to the interior surface of the support garment 50 by sewing. It is to be appreciated that the hook and loop fastener patches can be affixed to the support garment 50 by any suitable technique.

With reference to FIGS. 10, 13A, and 13B, according to an example of the present disclosure, the support garment 50 can be incorporated into a wearable cardiac therapeutic device with improved features for assembly of therapy electrodes 11 on the support garment 50.

The device includes a plurality of therapy electrodes 11 configured to deliver transcutaneous defibrillation shocks or transcutaneous pacing pulses to a patient's heart and the support garment 50 configured to support and hold the plurality of therapy electrodes 11 against the patient's body. The device can further include a plurality of ECG sensing electrodes 12 configured to sense ECG signals regarding a cardiac function of the patient. The support garment 50 can be configured to support and hold the plurality of ECG sensing electrodes 12 against the patient's body.

The support garment 50 includes a plurality of support pockets 56, 57 disposed on an inside surface of the support garment 50 for supporting the plurality of therapy electrodes 11 on the support garment 50 and a plurality of corresponding closure devices 61, such as a hook and loop fastener or other suitable closure devices. At least one closure device 61 is fastened to each of the plurality of support pockets 56, 57. The closure devices 61 are configured to facilitate opening and closing of the plurality of support pockets 56, 57 for assembly of the plurality of therapy electrodes 11 therein. It is to be appreciated that the closure device(s) 61 can be fastened to the support pockets 56, 57 in any suitable manner.

Aspects of the present disclosure are directed to monitoring and/or therapeutic medical devices configured to identify a patient physiological event and, in response to the identified event, to provide a notification to the patient wearing the device. The notification can include an instruction or request to perform a patient response activity. Successful completion of the patient response activity can cause the device to suspend or delay a device function, such as administering a treatment to a patient and/or issuing an alert or alarm.

In some examples, the medical device includes monitoring circuitry configured to sense physiological information of a patient. The controller can be configured to detect the patient physiological event based, at least in part, on the sensed physiological information. A patient event can be a temporary physiological problem or abnormality, which can be representative of an underlying patient condition. A patient event can also include injuries and other non-recurring problems that are not representative of underlying physiological condition of the patient. A non-exhaustive list of patient events that can be detected by an external medical device includes, for example: bradycardia, ventricular tachycardia (VT) or ventricular fibrillation (VF), atrial arrhythmias such as premature atrial contractions (PACs), multifocal atrial tachycardia, atrial flutter, and atrial fibrillation, supraventricular tachycardia (SVT), junctional arrhythmias, tachycardia, junctional rhythm, junctional tachycardia, premature junctional contraction, and ventricular arrhythmias such as premature ventricular contractions (PVCs) and accelerated idioventricular rhythm.

In some examples, the device controller is configured to notify the patient of the detection of the one or more events and to receive a patient response to the notification. The patient response can include performing a response activity identifiable by an input component associated with the medical device. In general, the response activity is selected to demonstrate or to provide information about the status of the patient and, in particular, to confirm that the patient is conscious and substantially aware of his or her surroundings. The response activity or activities can also be configured to confirm patient identity (e.g., that the person providing the response is the patient, rather than a bystander or impostor). The response activity can also demonstrate or test a patient ability such as one or more of psychomotor ability, cognitive awareness, and athletic/movement ability. In some examples, the response activity can be a relatively simple action, such as making a simple or reflexive movement in response to a stimulus applied by the device. In other examples, more complex activities, such as providing answers to questions requiring reasoning and logical analysis can be required. The device can be configured to select a particular response activity based on characteristics of the patient and/or the detected patient event.

In some examples, the device can instruct the patient to perform several actions that are each representative of patient ability. In other modes, the device can instruct the patient to perform different types of activities that are representative of different patient abilities. For example, the device can instruct the patient to perform a single activity requiring several patient abilities to complete correctly. Alternatively, the device can instruct the patient to perform a first activity representative of a first patient ability and, once the first activity is correctly completed, to perform a second activity representative of a second patient ability.

This disclosure relates to components, modules, subsystems, circuitry, and/or techniques for use in external medical devices. For example, such components, modules, subsystems, circuitry, and/or techniques can be used in the context of medical devices for providing treatment to and/or monitoring a patient. For example, such medical devices can include monitoring devices configured to monitor a patient to identify occurrence of certain patient events. In some implementations, such devices are capable, in addition to monitoring for patient conditions, of providing treatment to a patient based on detecting a predetermined patient condition.

In some examples, the medical device can be a patient monitoring device, which can be configured to monitor one or more of a patient's physiological parameters without an accompanying treatment component. For example, a patient monitor can include a cardiac monitor for monitoring a patient's cardiac information. Such cardiac information can include, without limitation, heart rate, ECG data, heart sounds data from an acoustic sensor, and other cardiac data. In addition to cardiac monitoring, the patient monitor can perform monitoring of other relevant patient parameters, including glucose levels, blood oxygen levels, lung fluids, lung sounds, and blood pressure.

FIGS. 14-16 illustrate an exemplary wearable medical device 100, such as a wearable defibrillator, which can incorporate the exemplary features of the support garment described in this disclosure.

The wearable medical device 100 includes a plurality of sensing electrodes 112 that can be disposed at various positions about the patient's body. The sensing electrodes 112 are electrically coupled to a medical device controller 120 through a connection pod 130. In some implementations, some of the components of the wearable medical device 100 are affixed to a garment 110 that can be worn on the patient's torso. According to an example of the present disclosure, the garment 110 shown in FIG. 14 can be the same as the support garment 50 discussed above with reference to FIG. 13A-13B.

The devices described herein are capable of continuous, substantially continuous, long-term and/or extended use or wear by, or attachment or connection to, a patient. In this regard, the device can be configured to be used or worn by, or attached or connected to, a patient, without substantial interruption, for example, up to hours or beyond (e.g., weeks, months, or even years). For example, in some implementations, such a period of use or wear can be at least 4 hours. For example, such a period of use or wear can be at least 24 hours or one day. For example, such a period of use or wear can be at least 7 days. For example, such a period of use or wear can be at least one month. In some implementations, such devices can be removed for a period of time before use, wear, attachment, or connection to the patient is resumed, e.g., to change batteries, to change or wash the garment, and/or to take a shower. Similarly, the device can be configured for continuous, substantially continuous, long-term and/or extended monitoring of one or more patient physiological conditions. For instance, in addition to cardiac monitoring, the medical device can be capable of monitoring a patient for other physiological conditions. Accordingly, in implementations, the device can be configured to monitor blood oxygen, temperature, glucose levels, sleep apnea, snoring and/or other sleep conditions, heart sounds, lung sounds, tissue fluids, etc. using a variety of sensors including radio frequency (RF) sensors, ultrasonic sensors, electrodes, etc. In some instances, the device can carry out its monitoring in periodic or aperiodic time intervals or times. For example, the monitoring during intervals or times can be triggered by a patient action or another event. For example, one or more durations between periodic or aperiodic intervals or times can be patient and/or other non-patient user configurable.

For example, as shown in FIG. 14, the controller 120 can be mounted on a belt worn by the patient. The sensing electrodes 112 and connection pod 130 can be assembled or integrated into the garment 110 as shown. The sensing electrodes 112 are configured to monitor the cardiac function of the patient (e.g., by monitoring one or more cardiac signals of the patient). While FIG. 14 shows four sensing electrodes 112, additional sensing electrodes can be provided, and the plurality of sensing electrodes 112 can be disposed at various locations about the patient's body.

The wearable medical device 100 can also optionally include a plurality of therapy electrodes 114 that are electrically coupled to the medical device controller 120 through the connection pod 130. The therapy electrodes 114 are configured to deliver one or more therapeutic transcutaneous defibrillating shocks, transcutaneous pacing pulses, and/or TENS pulses to the body of the patient if it is determined that such treatment is warranted. The connection pod 130 can include electronic circuitry and one or more sensors (e.g., a motion sensor, an accelerometer, etc.) that are configured to monitor patient activity. In some implementations, the wearable medical device 100 can be a monitoring-only device that omits the therapy delivery capabilities and associated components (e.g., the therapy electrodes 114). In some implementations, various treatment components can be packaged into various modules that can be attached or removed from the wearable medical device 100 as needed. As shown in FIG. 14, the wearable medical device 100 can include a patient interface pod 140 that is electrically coupled to, integrated in, and/or integrated with the patient interface of the medical device controller 120. For example, the patient interface pod 140 can include patient interface elements such as a speaker, a microphone responsive to patient input, a display, an interactive touch screen responsive to patient input, and/or physical buttons for input.

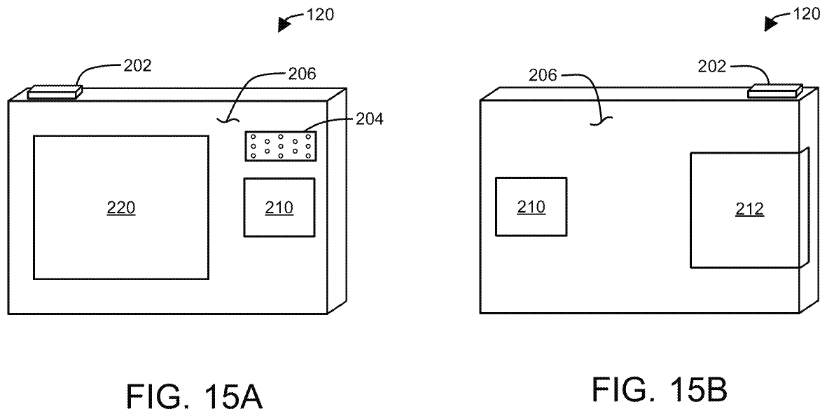

With reference to FIGS. 15A and 15B, an example of the medical device controller 120 is illustrated. The controller 120 can be powered by a rechargeable battery 212. The rechargeable battery 212 can be removable from a housing 206 of the medical device controller 120 to enable a patient and/or caregiver to swap a depleted (or near-depleted) battery 212 for a charged battery. The controller 120 includes a patient interface such as a touch screen 220 that can provide information to the patient, caregiver, and/or bystanders. In some implementations, in addition to or instead of a touch screen 220, the controller 120 can interact with the patient (e.g., receive patient input or provide information to the patient as described herein) via patient interface pod 140 (shown in FIG. 14). The patient interface pod 140 can be operatively coupled to the controller 120. In an example, the controller 120 can be configured to detect that if the patient interface pod 140 is operatively coupled to the controller 120, the controller 120 can then disable the patient interface elements of the controller 120 (e.g., touch screen 220) and instead communicate via the patient interface pod 140. The patient interface pod 140 can be wirelessly coupled with the controller 120. The patient interface pod 140 can take other forms and include additional functionality. For instance, the patient interface pod 140 can be implemented on a smartphone, tablet, or other mobile device carried by the patient. In another example, the patient interface pod 140 can be worn as a watch about the wrist of the patient, or as a band about an upper arm of the patient. In some implementations, the controller 120 can communicate certain alerts and information and/or be responsive to patient input via both the patient interface elements included in the controller 120 and the patient interface pod 140. The patient and/or caregiver can interact with the touch screen 220 or the patient interface pod 140 to control the medical device 100. The controller 120 also includes a speaker 204 for communicating information to the patient, caregiver, and/or the bystander. The controller 120 (and/or the patient interface pod 140) can include one or more response buttons 210. In some examples, when the controller 120 determines that the patient is experiencing cardiac arrhythmia, the speaker 204 can issue an audible alarm to alert the patient and bystanders to the patient's medical condition. In some examples, the controller 120 can instruct the patient to press one or both of the response buttons 210 to indicate that he or she is conscious, thereby instructing the medical device controller 120 to withhold the delivery of therapeutic defibrillating shocks. If the patient does not respond to an instruction from the controller 120, the medical device 100 can determine that the patient is unconscious and proceed with the treatment sequence, culminating in the delivery of one or more defibrillating shocks to the body of the patient. In some examples, as discussed in further detail herein, the controller 120 can additionally or alternatively instruct the patient to perform a response activity to indicate that he or she is conscious and further provide information to the controller 120 regarding the patient's status. For example, the controller 120 can instruct the patient to touch or manipulate the touch screen 220 or an interactive display on the patient interface pod 140 in a coordinated manner to confirm that he or she is conscious and has requisite awareness and/or psychomotor ability. In this way, the patient response confirms not only that buttons 210 were pressed, but that the patient is sufficiently conscious and aware to perform a response activity as instructed. The medical device controller 120 can further include a port 202 to removably connect sensing devices (e.g., ECG sensing electrodes 112) and/or therapeutic devices (e.g., therapy electrodes 114 shown in FIG. 14) to the medical device controller 120.

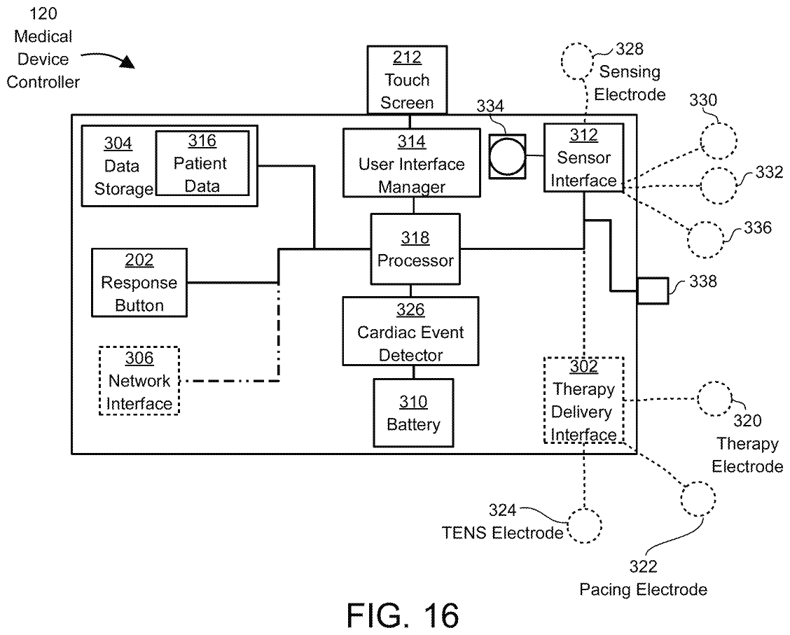

With reference to FIG. 16, a schematic example of the medical device controller 120 of FIGS. 14, 15A, and 15B is illustrated. As shown in FIG. 16, the controller 120 includes at least one processor 318, a patient interface manager 314, a sensor interface 312, an optional therapy delivery interface 302, data storage 304 (which can include patient data storage 316), an optional network interface 306, a patient interface 308 (e.g., including the touch screen 220 shown in FIGS. 15A and 15B), and a battery 310. The sensor interface 312 can be coupled to any one or combination of sensors to receive information indicative of cardiac activity. For example, the sensor interface 312 can be coupled to one or more sensing devices including, for example, sensing electrodes 328, contact sensors 330, pressure sensors 332, accelerometers or motion sensors 334, and radio frequency (RF)-energy based sensors 331 (e.g., tissue fluid sensors). The controller 120 can also include an optical sensor 336, such as a digital camera, for capturing static or video images of the device surroundings. Although designs from different vendors are different, a digital camera usually consists of a charge-coupled device (CCD) or complementary metal-oxide-semiconductor (CMOS) imaging sensor, a lens, a multifunctional video control chip, and a set of discrete components (e.g., capacitor, resistors, and connectors). The therapy delivery interface 302 (if included) can be coupled to one or more electrodes that provide therapy to the patient including, for example, one or more therapy electrodes 320, pacing electrodes 322, and/or TENS electrodes 324. The sensor interface 312 and the therapy delivery interface 302 can implement a variety of coupling and communication techniques for facilitating the exchange of data between the sensors and/or therapy delivery devices and the controller 120.

The medical device controller 120 can comprise one or more input components configured to receive a response input from the patient. The input components can comprise at least one of: the response button 210; the touch screen 220; an audio detection device, such as a microphone 338; the motion sensor 334; the contact sensor 330; the pressure sensor 332; a gesture recognitions component, such as the optical sensor 336; or a patient physiological sensor, such as the sensing electrodes 328.

In some examples, the medical device controller 120 includes a cardiac event detector 326 to monitor the cardiac activity of the patient and identify cardiac events experienced by the patient based on received cardiac signals. In other examples, cardiac event detection can be performed using algorithms for analyzing patient ECG signals obtained from the sensing electrodes 328. Additionally, the cardiac event detector 326 can access patient templates (e.g., which can be stored in the data storage 304 as patient data 316) that can assist the cardiac event detector 326 in identifying cardiac events experienced by the particular patient (e.g., by performing template matching algorithms).

The at least one processor 318 can perform a series of instructions that control the operation of the other components of the controller 120. In some examples, the patient interface manager 314 is implemented as a software component that is stored in the data storage 304 and executed by the at least one processor 318 to control, for example, the patient interface component 308. The patient interface manager 314 can control various output components and/or devices of the medical device controller 300 (e.g., patient interface 220 and/or patient interface pod 140 shown in FIG. 14) to communicate with external entities consistent with various acts and/or display screens described herein. For example, such output components and/or devices can include speakers, tactile and/or vibration output elements, visual indicators, monitors, displays, LCD screens, LEDs, Braille output elements, and the like. Additionally, the patient interface manager 314 can be integrated with the treatment-providing components of the controller 120 so that the patient can control and, in some cases, suspend, delay, or cancel treatment using the patient interface.

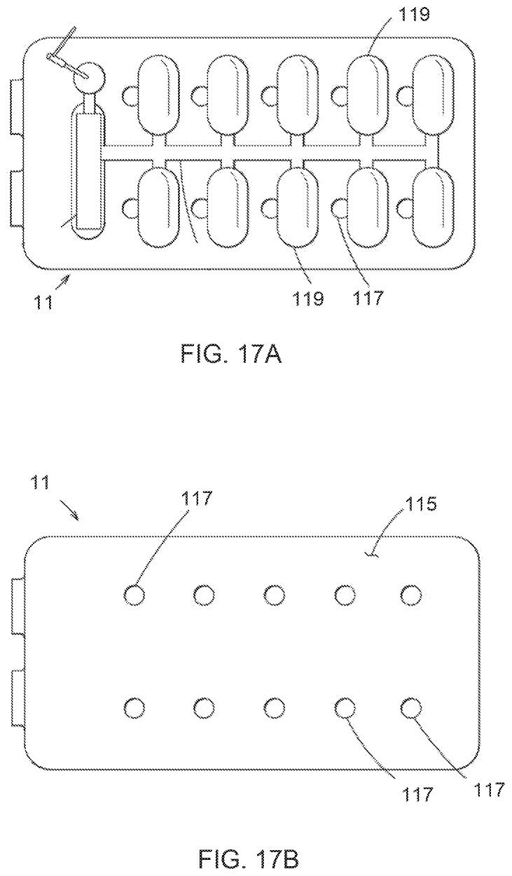

FIGS. 17A and 17B illustrate the therapy electrode 11 utilized in the various examples described herein. The therapy electrode 11 includes a conductive bottom surface 115 configured to establish an electrical interface with the patient's skin through the pocket 57. The conductive bottom surface 115 can be metallic. The conductive bottom surface 115 can include one or more apertures 117 through which a conductive gel can be dispensed to improve the electrical interface between the conductive bottom surface 115 and the patient's skin. The conductive gel can be dispensed from one or more gel packs 119 arranged in fluid communication with the apertures 117 on a side of the conductive bottom surface 115 facing away from the patient. The controller 120 (shown in FIG. 16) can control dispensing of the conductive gel from the gel packs 119.

Although a wearable medical device and a support garment for such a device have been described in detail for the purpose of illustration based on what is currently considered to be the most practical examples, it is to be understood that such detail is solely for that purpose and that the subject matter of this disclosure is not limited to the disclosed examples, but, on the contrary, is intended to cover modifications and equivalent arrangements. For example, it is to be understood that this disclosure contemplates that, to the extent possible, one or more features of any example can be combined with one or more features of any other example.

1. A non-invasive wearable ambulatory cardiac defibrillator, comprising:
a garment configured to be worn around a torso of a patient;
at least one sensing electrode attached to the garment and configured to sense electrical signal(s) at the surface of the patient's skin indicative of electrical activity of the patient's heart;
two or more therapy electrodes attached to the garment and configured to deliver one or more defibrillation pulses to the patient, wherein the two or more therapy electrodes comprise:
an anterior therapy electrode configured to be disposed on an anterior portion of the patient's body; and
at least two posterior therapy electrodes configured to be disposed on a posterior portion of the patient's body; and
a controller in communication with the at least one sensing electrode and the therapy electrodes, the controller configured to receive the electrical signal(s) from the at least one sensing electrodes and to cause delivery of the one or more defibrillation pulses from two or more therapy electrodes based on the controller detecting a cardiac arrhythmia in the received electrical signal(s);
at least one strap attached to a back portion of the garment, the at least one strap exerting a normal force on the at least two posterior therapy electrodes to exert a substantially uniform normal force over the surfaces of the at least two posterior therapy electrodes and/or limit displacement of the at least two posterior therapy electrodes.

2. The defibrillator of claim 1, wherein the at least one strap is attached to an external surface of the garment.

3. The defibrillator of claim 1, wherein the at least one strap comprises:
a first strap extending from a right shoulder portion of the garment to a left waist portion of the garment; and
a second strap extending from a left shoulder portion of the garment to a right waist portion of the garment such that the second strap crosses the first strap.

4. The defibrillator of claim 1, where the at least one strap is adjustable in length, wherein adjustment of the length of the at least one strap adjusts the normal force exerted on the at least two posterior therapy electrodes.

5. The defibrillator of claim 1, wherein the at least one strap comprises:
a first strap extending from a right shoulder portion of the garment to a left shoulder portion of the garment;
a second strap extending from the first strap to a right waist portion of the garment; and
a third strap extending from the first strap to a left waist portion of the garment.

6. The defibrillator of claim 5, wherein the first strap comprises a curved portion extending over the at least one therapy electrode.

7. The defibrillator of claim 1, wherein the at least one strap comprises:
a central strap extending at least partially over the at least one therapy electrode;
a first strap extending from a right shoulder portion of the garment to the central strap;
a second strap extending from a left shoulder portion of the garment to the central strap;
a third strap extending from a right waist portion of the garment to the central strap; and
a fourth strap extending from a left waist portion of the garment to the central strap.

8. The defibrillator of claim 1, wherein the at least one strap is disposed internally within the garment.

9. The defibrillator of claim 1, wherein one of the garment and the at least one strap comprises a plurality of loops, wherein the other of the garment and the at least one strap comprises a hook configured to engage any of the plurality of loops to adjust a tautness of the at least one strap.

10. The defibrillator of claim 1, wherein the two or more therapy electrodes are configured to deliver a biphasic shock to the patient.

11. The defibrillator of claim 1, wherein the two or more therapy electrodes are configured to deliver pacing pulses to the patient.

12. The defibrillator of claim 1, wherein the controller is configured to monitor for at least one of ventricular fibrillation or a ventricular tachycardia event.

13. (canceled)

14. The defibrillator of claim 1, wherein the garment is configured to be separable from the at least one sensing electrode and the two or more therapy electrodes.

15. The defibrillator of claim 1, wherein the controller is configured to generate ECG information from the electrical signal(s) received from the at least one sensing electrode and to cause delivery of the one or more therapeutic pulses from the at least one therapy electrode.

16-23. (canceled)

24. A non-invasive wearable ambulatory cardiac defibrillator, comprising:
a garment configured to be worn around a torso of a patient;
at least one sensing electrode attached to the garment and configured to sense electrical signal(s) at the surface of the patient's skin indicative of electrical activity of the patient's heart;
two or more therapy electrodes attached to the garment and configured to deliver one or more defibrillation pulses to the patient, wherein the two or more therapy electrodes comprise:
an anterior therapy electrode configured to be disposed on an anterior portion of the patient's body; and
at least two posterior therapy electrodes configured to be disposed on a posterior portion of the patient's body;
a controller in communication with the at least one sensing electrode and the therapy electrodes, the controller configured to receive the electrical signal(s) from the at least one sensing electrodes and to cause delivery of the one or more defibrillation pulses from two or more therapy electrodes based on the controller detecting a cardiac arrhythmia in the received electrical signal(s); and
a panel attached to a back portion of the garment at least partially over the at least two posterior therapy electrodes, the panel exerting a normal force on the at least two posterior therapy electrodes to exert a substantially uniform normal force over the surfaces of the at least two posterior therapy electrodes and/or limit displacement of the at least two posterior therapy electrodes,
wherein the panel is made from a material that is less elastic than a material of the back portion of the garment.

25. The defibrillator of claim 24, wherein the panel is attached to the back portion of the garment with an adhesive.

26-27. (canceled)

28. The defibrillator of claim 24, wherein the panel comprises:
a central section at least partially covering the at least one therapy electrode;
a first strip extending from the central section to a right waist portion of the garment; and
a second strip extending from the central section to a left waist portion of the garment.

29. The defibrillator of claim 24, wherein the two or more therapy electrodes are configured to deliver at least one of a biphasic shock to the patient or pacing pulses to the patient.

30. (canceled)

31. The defibrillator of claim 24, wherein the controller is configured to monitor for at least one of ventricular fibrillation or a ventricular tachycardia event.

32. (canceled)

33. The defibrillator of claim 24, wherein the garment is configured to be separable from the at least one sensing electrode and the two or more therapy electrodes.

34. The defibrillator of claim 24, wherein the controller is configured to generate ECG information from the electrical signal(s) received from the at least one sensing electrode and to cause delivery of the one or more therapeutic pulses from the at least one therapy electrode.

35. (canceled)
